(19) United States
(12) Patent Application Publication
(10) Pub. No.: US 2007/0243156 A1
(43) Pub. Date: Oct. 18, 2007

(54) Synthesis and utilization of peptide mimetics in drug discovery and medicine

(76) Inventor: Mitra Tadayoni-Rebek, La Jolla, CA (US)

Correspondence Address: Mitra Tadayoni-Rebek, 2330 Calle Del Oro, La Jolla, CA, 92037-3034, US

(21) Appl. No.: 11/374,294

(22) Filed: Nov. 5, 2004

(51) Int. Cl. A61K 38/16; A61K 31/785; C08L 89/00; C08L 75/02

(52) U.S. Cl. 424/78.27; 514/12; 530/300; 530/350; 525/54.1; 525/440.06

(57) ABSTRACT

The disclosed embodiments are directed to synthesis of peptidomimetic compounds and their utilization in drug discovery and medicine. The disclosed compounds high structural similarities to naturally occurring peptides, but having stability to enzymes and greater bioavailability through their ability to cross biological membranes.

### BACKGROUND OF THE INVENTION

1. Field of the Invention

The invention relates generally to synthesis of peptidomimetic compounds and their utilization in drug discovery and medicine. In particular, the invention relates to synthetic molecules with high structural similarities to naturally occurring peptides, but having stability to enzymes and greater bioavailability through their ability to cross biological membranes.

2. Related Art

The information contained in this section relates to the background of the art of the present invention without any admission as to whether or not it legally constitutes prior art.

Peptides offer enormous structural diversity and are easy to obtain in vast numbers through combinatorial synthesis or biological methods such as phage display. They provide lead compounds for medicinal chemistry efforts but there is no direct path between peptide leads and non-peptide pharmaceuticals.

Peptides suffer from poor bioavailability; they typically resist crossing membranes and have short life-times through degradation by enzymes present in living systems.

Peptide mimetics are molecules that overcome these limitations. There are hundreds of β-turn mimetics and even some α-helix and β-sheet mimetics.

### SUMMARY OF THE INVENTION

The use of peptides as drugs has not only been limited by their natural susceptibility to proteolytic degradation, but also by the general difficulty of delivery inside cells. The invention includes a general method of converting “retro-inverso” peptides and peptides to synthetic molecules while maintaining the molecular shape required for recognition and binding to their cellular targets.

The invention provides a method to overcome shortcomings associated with peptide as therapeutics.

The work of Goodman (M. Goodman, and M. Chorev. 1979, Acc. Chem. Res. 12, p 1-7; M. Chorev and M. Goodman, 1993, Acc. Chem. Res. 26, p 266-273.) teaches that a retro-inversopp sequence can be prepared wherein the synthetic peptide backbone runs in the opposite sense—carbonyl, α-carbon, nitrogen- to the naturally occurring peptide-nitrogen, α-carbon, carbonyl. In sequence the stereochemistry at the α-carbon is also inverted (D vs the naturally occurring L configuration of the amino acid). The retro-inverso sequence places the side chains in a manner that can be superimposed on the original L peptide. It is shown in FIG. 1 that these retro-inverso peptides place the hydrogen bond donors and acceptors of the backbone amide in precisely the wrong position to imitate the original.

One aspect of this invention provides reduction of retro-inverso peptides. Where “reduction” refers to conversion of the carbonyl groups of amide linkages to CH₂ in the presence of reducing agents, e.g., hydrides.

In a preferred embodiment of this aspect of invention, the secondary amino groups in the reduced retro-inverso peptides are modified with activated carbonyls to form new molecules that can achieve the same shapes as the corresponding peptides but can vary in solubility characteristics, FIG. 2. For examples, reactions with isocyanates give ureas, with chloroformates give carbamates (urethanes), with thiocyanates give thioureas and with cyanogens halides give cyanamides. Preferred examples include the following structures:

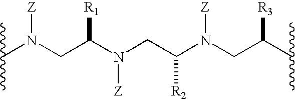

In the above structure, Z is a group independently selected at each occurrence from the group consisting of —COR, —COOR, —CONHR, —CSNHR, where R is selected from a group consisting of —H, methyl, ethyl, and benzyl, R₁, R₂, and R₃ are amino acid side chains.

In this aspect of the invention, acylation of the amnio groups of the reduced retro-inverso peptides will recover the appropriate donor and acceptor properties of the original peptide. Preferred example includes the following structure:

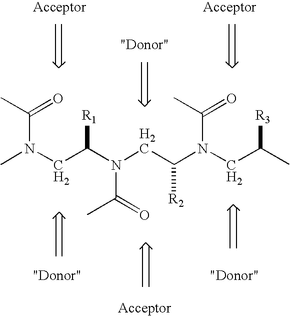

In the above structure, the carbonyl acceptors of the mimic are in positions close to those of the original peptide and the NH donors are replaced by a CH₂ group. The CH₂ has been shown to provide a complement to those acceptors on the target, e.g. enzyme or receptor, that feature uncharged hydrogen bond acceptors (P. A. Bartlett, C. K. Marlow, Science, 1987, 235, p 569-571; D. H. Tronrud, H. M. Holden, B. W. Matthews Science, 1987, 235, p 571-574; K. M. Mertz, P. A. Kollman, J. Am. Chem. Soc. 1989, 111, p 5648-5658; and Alan R. Fersht, Trends in Biochemical Science, 1987, 12, p 301-304 and references therein).

Another aspect of the invention is directed to reduction of peptides-removing the amide carbonyls of the peptide backbone that are responsible for biodegradation and transport limitations. The amides can be reduced to amines in the presence of reducing agents, e.g., hydrides, and new carbonyls are introduced on these atoms as side chains. Acylation or carbamylation of the amines creates structures that are closely related in shape and function to the original peptides. That is, they present the same amino acid side chains responsible for their activity, yet resist enzymatic degradation and can readily cross biological membranes.

In a preferred embodiment of this aspect, a method follows existing experimental procedures (Y. S. Oh, T. Yamazaki and M. Goodman, Macromolecules 1992, 25, p 6322-6331) for reduction/acylation of polyamino acids in solution. The method also comprises the reduction of peptides on solid supports and acylation in solution following their liberation from the support (S. Manku, C. Laplante, D. Kopac, T. Chan and D. G. Hall J. Org. Chem. 2001, 66, p 874-885; A. Nefzi, J. M. Ostresh and R. A. Houghton, Tetrahedron 1999, 55, p 335-344). The methods involving synthesis from naturally occurring peptides are summarized in FIG. 3.

In methods mentioned above, the reaction of the amino groups resulting from the reduction with a number of activated carbonyls is possible. Such functionalization generates new molecules that can achieve the same shapes as the corresponding peptides but can vary in solubility characteristics. For examples, reactions with isocyanates give ureas, with chloroformates give carbamates (urethanes), with thiocyanates give thioureas and with cyanogens halides give cyanamides.

In another aspect of this invention, an entirely new method, involving sequential synthesis from amino alcohols is provided, FIG. 4. The method is compatible with all naturally-occurring amino acid side chains including those (aspartic acid, asparagines, glutamic acid, glutamine) that cannot withstand direct reduction of peptides.

In the preferred embodiment of this aspect, the reduced retro/inverso peptide can be synthesized from the respective D-amino alcohols as in the case of the L-amino alcohols as described above and as shown in FIG. 4.

In the method shown in FIG. 4, the reaction of the amines with a number of activated carbonyls is possible and gives new molecules that can achieve the same shapes as the corresponding peptides but can vary in solubility characteristics. For examples, reactions with isocyanates give ureas, with chloroformates give carbamates (urethanes), with thiocyanates give thioureas and with cyanogens halides give cyanamides.

A preferred structure for peptide mimetic compounds in this invention involves β-strands that are often found in loops that connect other secondary structures such as helices.

This invention includes cyclic peptide templates as well as linear peptide templates.

### BRIEF DESCRIPTION OF THE DRAWINGS

FIG. 1 shows peptide, retro/inverso peptide and retro/inverso peptide mimetics obtained by reduction of corresponding peptide.

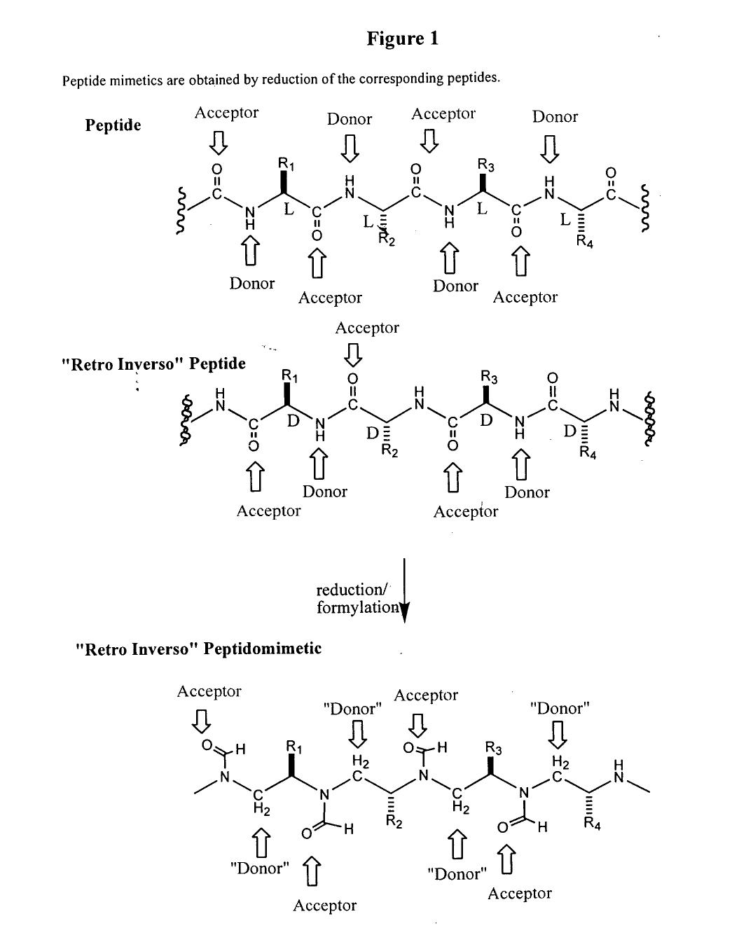

FIG. 2 illustrates modification of secondary amines in peptide mimetics with activated carbonyls.

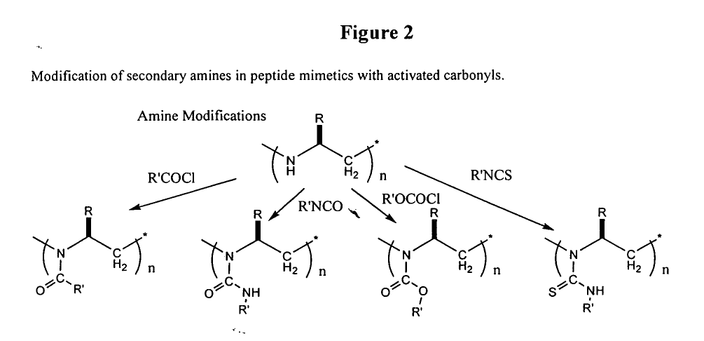

FIG. 3 illustrates synthesis of peptide mimetics by reduction of naturally occurring peptides followed by formylation.

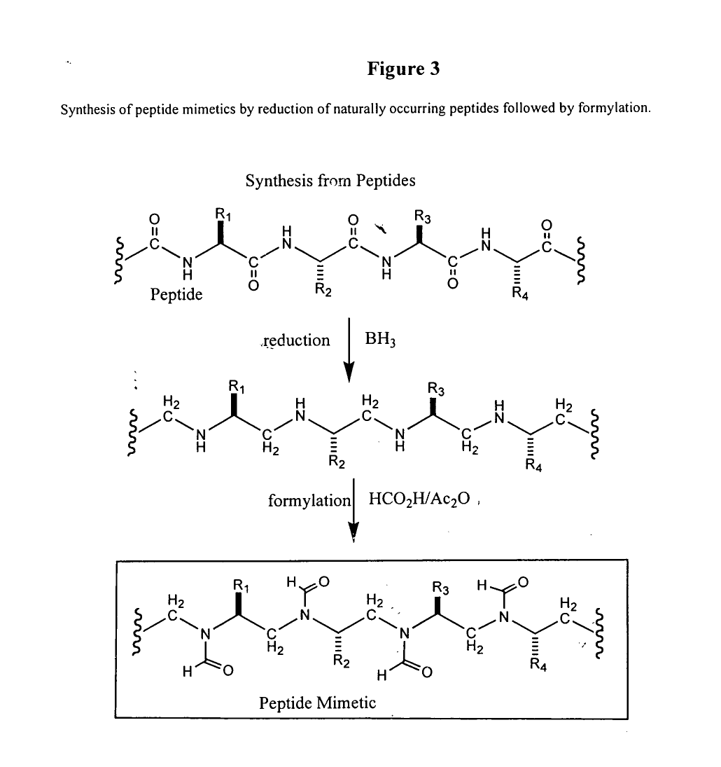

FIG. 4 depicts stepwise synthesis of peptide mimetics from amino alcohols followed by formylation.

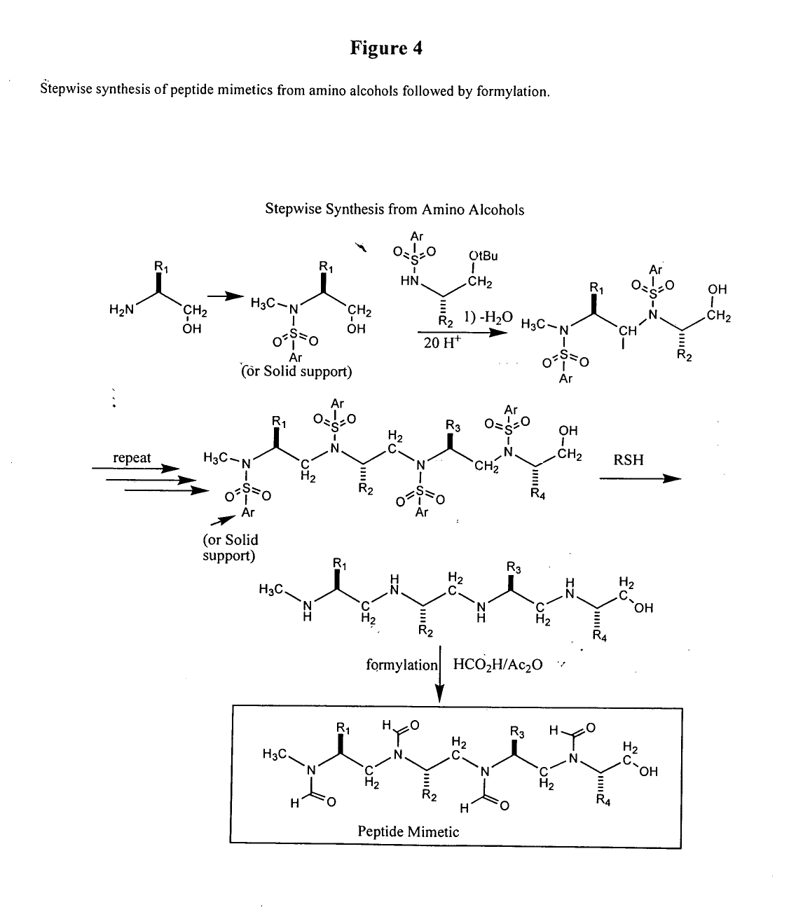

### DESCRIPTION OF CERTAIN EMBODIMENTS OF THE INVENTION

The present invention provides a method for preparing peptide mimetics that are superior to the existing β-strand mimetics because of the ease of synthesis (A. B. Smith, III, R. Hirschmann, A. Pasternak, W. Yao, and P. A. Sprengeler, J. Med. Chem. 1997, 40, 2440-2444; A. B. Smith, III, S. D. Knight, P. A. Sprengeler, and R. Hirschmann, Bioorganic and medicinal chemistry, 1996, Vol. 4, No. 7, 1021-1034), the resistance to both proteases and aggregation, and improved membrane crossing characteristics.

We claim:

1. A compound obtained from reduction of a peptide.

2. A compound according to claim 1, wherein said compound maintains the molecular shape required for recognition and binding to biological targets.

3. A compound according to claim 1, wherein said compound is therapeutic.

4. A compound according to claim 1, wherein said compound is used as therapeutics in mammals.

5. A compound according to claim 1, wherein said compound is used in drug discovery.

6. A compound according to claim 1, wherein said compound is formed from peptides comprising up to 10 amino acids.

7. A compound according to claim 1, wherein said compound is formed from peptides comprising up to 20 amino acids.

8. A compound according to claim 1, wherein said compound is formed from peptides comprising up to 100 amino acids.

9. The compound of claim 1, wherein said compound has the following general structure: 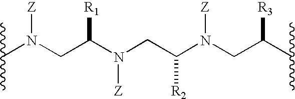
Wherein:
R₁, R₂, and R₃ is any amino acid side chain.
Z is a group independently selected at each occurrence from the group consulting —CO—H, —COCH3, —COOCH3, —CONHR, —CSNHR.

10. The compound of claim 9, wherein said compound is generated by reducing a retro-inverso peptide.

11. A method for preparing compound of claim 9 represented by the following scheme: 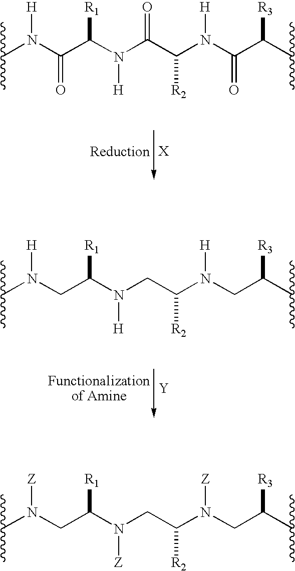
Wherein:
X is a reducing agent, e.g., borane/tetrahydrofurane, lithium aluminum hydride.
Y is a compound independently selected at each occurrence, e.g., acetic acid, chloroformate, isocyanate, isothiocyanate.
R₁, R₂, and R₃ is any amino acid side chain.
Z is a group independently selected at each occurrence from the group consisting —CO—H, —COCH3, —COOCH3, —CONHR, —CSNHR

12. The compound of claim 1, wherein said compound has the following general structure: 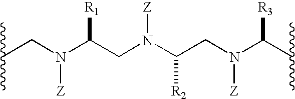
Wherein:
R₁, R₂, and R₃ is any amino acid side chain.
Z is a group independently selected at each occurrence from the group consisting —COH, —COCH3, —COOCH3, —CONHR, —CSNHR.

13. The compound of claim 12, wherein said compound is formed by reducing a linear peptide.

14. A method for preparing compound of claim 12 according to the following scheme: 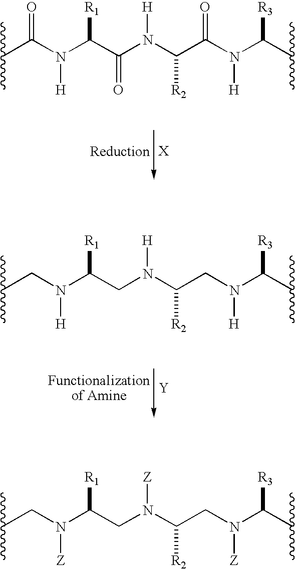
Wherein:
X is a reducing agent, e.g., borane, lithium aluminum hydride.
Y is a compound independently selected at each occurrence, e.g., formic acid, acetic acid, chloroformate, isocyanate, isothiocyanate.
R₁, R₂, and R₃ is any amino acid side chain.
Z is a group independently selected at each occurrence from the group consisting —COH, —COCH3, COOCH3, CONHR, CSNHR

15. A method for preparing compound of claim 1, includes the following steps:
Where
Stepwise Synthesis from Amino Alcohols 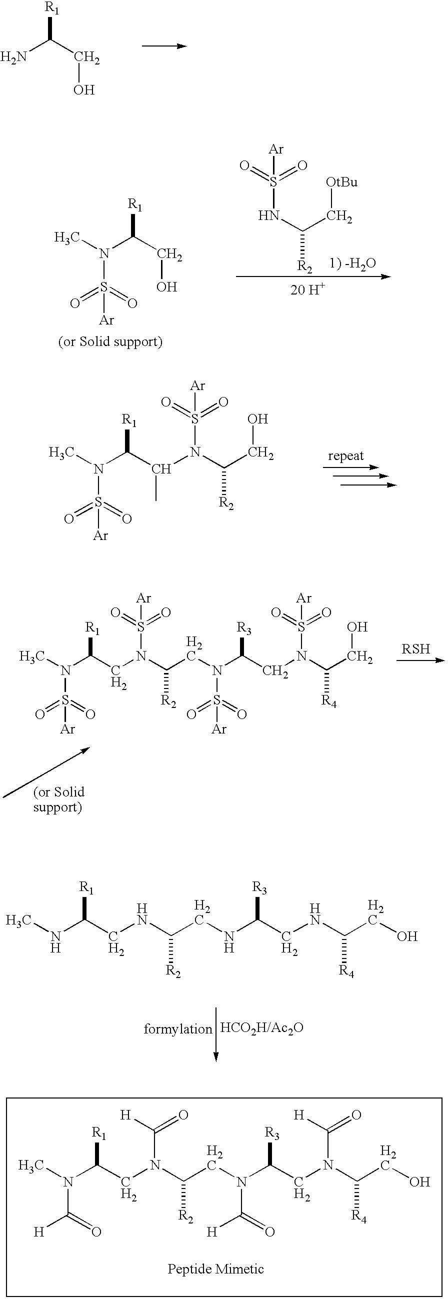
Wherein:
R₁, R₂, R₃, and R₄ are amino acid side chains.
Ar is aromatic group.

16. The compound of claim 1, wherein said peptide is a cyclic peptide.

17. The compound claim 1, wherein said compound is covalently linked to a fluorescent probe.
